(19) United States
(12) Plant Patent Application Publication
(10) Pub. No.: US 2002/0148023 P1
(43) Pub. Date: Oct. 10, 2002

(54) Chrysanthemum named Amor White

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/734,606

(22) Filed: Dec. 13, 2000

(51) Int. Cl. A01H 5/00

(52) U.S. Cl. PLT/294

(57) ABSTRACT

A chrysanthemum plant named AMOR WHITE characterized by its medium sized blooms with white ray florets and prolific branching; natural season flower date September 4-9; blooming for a period of 7 weeks.

### RELATED APPLICATIONS

| Application no. | Filing date | Title |
|---|---|---|
| | | AMOR |
| | | AMOR CORAL |

### BACKGROUND OF THE INVENTION

The new plant of the present invention comprises a new and distinct cultivar of Chrysanthemum plant that is a natural occuring sport of a parent chrysanthemum named AMOR. A comparison with Parent chrysanthemum AMOR is also made in this application. The new cultivar was discovered as a whole plant mutation in September 1998 by Rob Noodelijk on a cultivated field in Rijsenhout Holland. The first act of asexual reproduction of AMOR WHITE was accomplished when vegetative cuttings were taken from the initial selection in October 1998 in Rijsenhout Holland.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

FIG. 2 shows the various stages of bloom and foliage of the new cultivar.

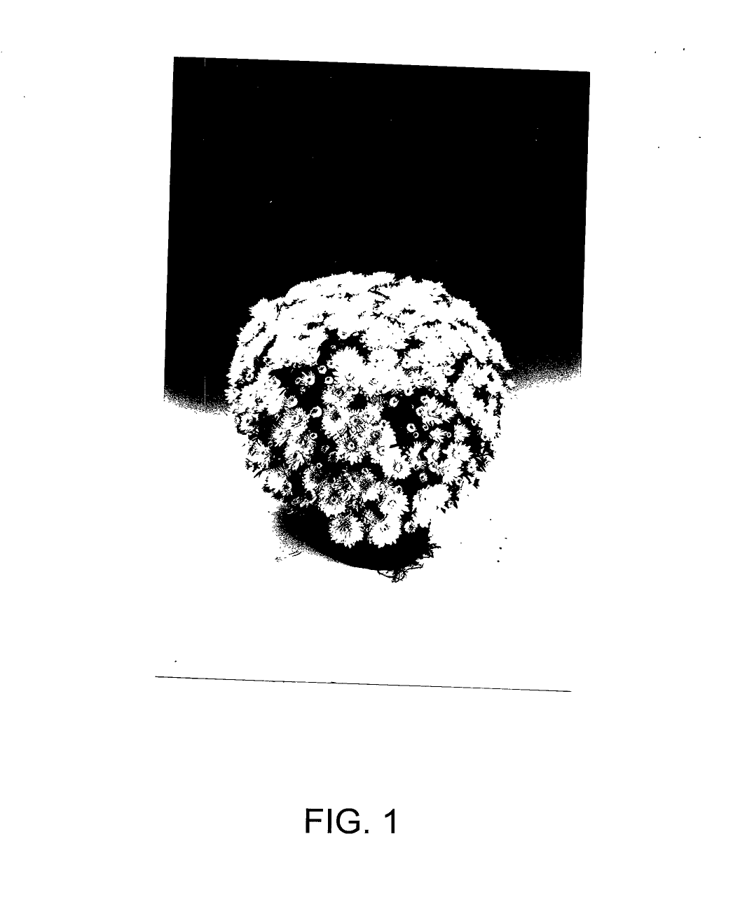

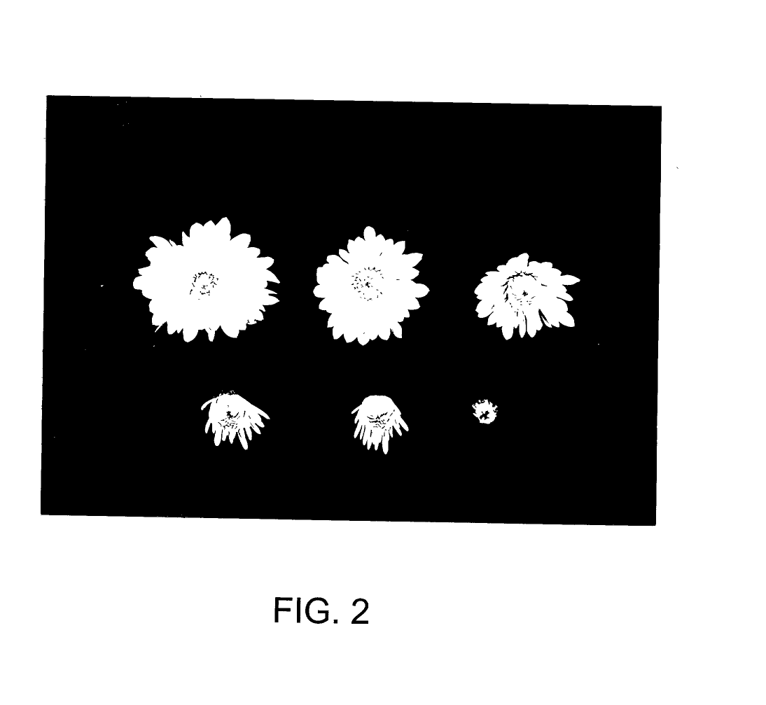

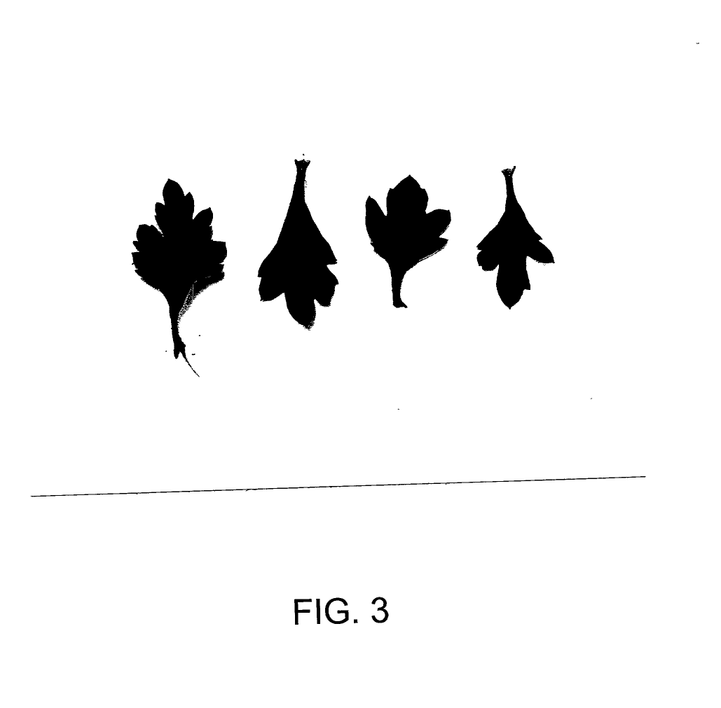

### DESCRIPTION OF THE INVENTION

This new variety of chrysanthemum is of the botanical classification Dendranthema Grandiflora. The observations and measurements were gathered from plants grown out door in Rijsenhout, Holland under natural day length and temperature and planted week 22 in 1999 and 2000. The natural blooming date of this crop was September 4-9 (week 36). The average height of the plants was 32-38 cms. This new variety produces medium sized blooms with white ray florets and a cream white center blooming for a period of 7 weeks

When AMOR, AMOR WHITE and AMOR CORAL are being compared the following differences and similarities are noticed: The differences of AMOR and AMOR WHITE are (1) Flower color and (2) Vigour. All other characteristics of AMOR and AMOR WHITE are similar

(1) Flower color. AMOR WHITE is a color mutant of AMOR, bearing white blooms instead of pink blooms, AMOR CORAL has coral blooms.

(2) Vigour: The plants of AMOR WHITE tend to be a little more vigorous. AMOR CORAL is a litttle less vigorous. These differences are very small.

The following is a description of the plant and characteristics that distinguish AMOR WHITE as a new and distinct variety.

The color designations are taken from the plant itself. Accordingly, any discrepancies between the color designations and the colors depicted in the photographs are due to photographic tolerances.

Botanical Classification

| CULTIVAR | | | AMOR |
|---|---|---|---|
| AMOR WHITE | | AMOR | CORAL |
| Bud | | | |
| Size | medium, cross- | | |
| | section +/−1.2 cm | | |
| | height +/−1.0 cm | | |
| Outside Color | White 155A | Red-purple | Red-Purple |
| | 74 C | 60D | |
| Bloom | | | |
| Size | Medium | | |
| Fully Expanded | 5.0-5.5 | | |
| Borne(number of | Upper and lower | | |
| blooms per | portion plural | | |
| branch) | blooms per | | |
| | branch (approx. | | |
| | 5 blooms | | |
| | per branch) | | |
| Length of lateral | From top till | From top till | From top till |
| branch | bottom 14-15 cm | bottom 12- | bottom 12- |
| | | 13 cm | 13 cm |
| Lateral branch, | Weak | | |
| Attachment | | | |
| branching | Mounding and | | |
| | prolific with 8-10 | | |
| | breaks after | | |
| | pinching | | |
| Form | Decorative | | |
| Performance (on | 7 weeks | | |
| the plant) | | | |
| Color | | | |
| Center of Flower | Immature | Immature | Immature |
| | White 155A | red-purple | red-purple |
| | | 74B | 60D |
| Color of the | White 155D | Red-purple | Red-purple |
| upper surface of | | 74C | 63C |
| the Majority of | | | |
| the ray-florets | | | |
| Color of the | White 155D | Red-purple | Red-purple |
| lower surface of | | 74D | 62C |
| the Majority of | | | |
| the ray-florets | | | |
| Tonality from | A mounded | | |
| Distance | decorative | | |
| | garden mum with | | |
| | white blooms | | |
| Discoloration | None | To red- | To Red- |
| | | purple 74D | purple 62C |
| Ray florets | | | |
| Texture | Upper side and | | |
| | under side smooth | | |
| Number | 200-230 | | |
| Cross-section | Concave | | |
| Longitudinal axis | Incurved to | | |
| of the majority | straight | | |
| Shape of tip | Pointed | | |
| Fragrance | Typical | | |
| | chrysanthemum | | |
| Disc Diameter | No disc floret | | |
| (natural season | present | | |
| crop when | | | |
| visible) | | | |
| Reproductive | | | |
| Organs | | | |
| Stamen | Not present | | |
| Pollen | None | | |
| Styles (present in | Green, thin | | |
| both ray and disc | | | |
| florets) | | | |
| Style Length | +/−4 mm | | |
| Stigmas | Yellow-green | | |
| Stigma Width | +/−1 mm | | |
| Ovaries | Enclosed in calyx | | |
| Plant | | | |
| Form | A garden mum | | |
| | (pot mum) meant | | |
| | for out door use | | |
| Growth | Medium | | |
| Height (from | 32-38 cms | 32-36 cms | 30-35 cms |
| soil level) | | | |
| Stem Color | Green 138 B | | |
| Stem Strength | Strong | | |
| Stem Brittleness | Present | | |
| Stem | Absent | Present | Present |
| Anthocyanin | | | |
| Coloration | | | |
| Natural blooming | 4-9 September | | |
| date | | | |
| Foliage | | | |
| Color | Upper side green | | |
| | 138 A | | |
| | Under side green | | |
| | 138 B | | |
| Size | small, | | |
| | length +/−6 cm | | |
| | width +/−5 cm | | |
| Quantity (number | 12-14 | | |
| per lateral branch) | | | |
| Shape | Oval | | |
| Texture | Fleshy | | |
| Ribs and Veins | Ribs and veins | | |
| | not so well | | |
| | developed | | |
| Edge | Crenated | | |
| Shape of Base of | Acute | | |
| Sinus Between | | | |
| Lateral Lobes | | | |
| Margin of Sinus | Diverging | | |
| Between Lateral | | | |
| Lobes | | | |
| Shape of Base | Asymmetric | | |
| Apex | Mucronate | | |

I claim:

1. A new and distinc variety of chrysanthemum plant as described and illustrated
